(19) United States
(12) Patent Application Publication
(10) Pub. No.: US 2023/0165491 A1
(43) Pub. Date: Jun. 1, 2023

(54) METHOD, MONITORING SYSTEM AND MONITORING SYSTEM DEVICE FOR MONITORING BLOOD OXYGEN SATURATION OF USER, AND MACHINE-READABLE STORAGE MEDIUM

(71) Applicant: National Yang Ming Chiao Tung University, Taipei City (TW)

(72) Inventor: Albert Chihchieh YANG, Taipei City (TW)

(21) Appl. No.: 17/806,049

(22) Filed: Jun. 8, 2022

(30) Foreign Application Priority Data
Nov. 30, 2021 (TW) 110144656

(51) Int. Cl. A61B 5/145; A61B 5/00; G16H 40/63; G16H 50/30

(52) U.S. Cl. CPC A61B 5/14542; A61B 5/7282; A61B 5/746; A61B 5/7275; G16H 40/63; G16H 50/30

(57) ABSTRACT

A method for monitoring blood oxygen saturation level of a user includes: obtaining a plurality of values of blood oxygen saturation level measured during a predetermined time duration; obtaining an n^{th} degree polynomial equation that fits the values of blood oxygen saturation level; determining whether a leading coefficient of the n^{th} degree polynomial equation is smaller than a predetermined value that is associated with a normal variation in blood oxygen saturation level; and when it is determined that the leading coefficient is smaller than the predetermined value, outputting a warning indicating that the blood oxygen saturation level of the user is getting worse.

### CROSS-REFERENCE TO RELATED APPLICATION

This application claims priority of Taiwanese Patent Application No. 110144656, filed on Nov. 30, 2021.

### FIELD

The disclosure relates to a method for monitoring physiological information, more particularly to a method for monitoring blood oxygen saturation level of a user.

### BACKGROUND

After the global outbreak of Coronavirus disease 2019 (COVID-19), the number of COVID-19 patients grows rapidly. About 20-40% of COVID-19 patients have experienced silent hypoxia (also referred to as happy hypoxia). Patients with silent hypoxia have low blood oxygen saturation level but do not show symptoms of respiratory distress, and therefore it is likely that they are not sent to the intensive care unit in time.

To avoid the above-mentioned situation, a potential COVID-19 patient under quarantine at home, in a government quarantine facility, or in a quarantine hotel may use a wearable blood oxygen saturation detector, such as a pulse oximeter, to measure his/her blood oxygen saturation level. When the measured blood oxygen saturation level falls below 94%, the patient should raise alert and even go to a hospital for further diagnosis and treatment.

In addition, to accurately determine whether the blood oxygen saturation level is getting worse, continuous observation of the blood oxygen saturation level for an extended period of time, such as 48 hours, is usually required, and a doctor or a medical professional has to make the determination according to the observed blood oxygen saturation level using his/her professional knowledge.

### SUMMARY

Therefore, an object of the disclosure is to provide a method for monitoring blood oxygen saturation level of a user.

According to one embodiment of the disclosure, the method is implemented by a monitoring device communicating with a blood oxygen saturation detector, and the blood oxygen saturation detector is worn by she user to periodically measure the blood oxygen saturation level of the user. The method includes steps of:

obtaining a plurality of values of blood oxygen saturation level from the blood oxygen saturation detector, the values of blood oxygen saturation level being measured during a time period of a predetermined duration;

obtaining an n^{th} degree polynomial equation in two variables that represents a curve which fits the values of blood oxygen saturation level, where n is an integer equal to or greater than one;

determining whether a leading coefficient of the n^{th} degree polynomial equation smaller than a predetermined value that is associated with a normal variation in blood oxygen saturation level; and

when it is determined that the leading coefficient is smaller than the predetermined value, outputting a warning indicating that the blood oxygen saturation level of the user is getting worse.

Another object of the disclosure is to provide a monitoring system for monitoring blood oxygen saturation level of a user. The monitoring system includes a blood oxygen saturation detector and a monitoring device. The blood oxygen saturation detector is adapted to be worn by the user, and is configured to periodically measure the blood oxygen saturation level of the user and to output a value of blood oxygen saturation level for each measurement of the blood oxygen saturation level. The monitoring device is configured to communicate with the blood oxygen saturation detector to obtain a plurality of values of blood oxygen saturation level from the blood oxygen saturation detector, where the values of blood oxygen saturation level were measured in a time period of predetermined duration. The monitoring device is further configured to obtain an n^{th} degree polynomial equation in two variables that represents a curve which fits the values of blood oxygen saturation level, where n is an integer equal to or greater than one, determine whether a leading coefficient of the n^{th} degree polynomial equation is smaller than a predetermined value that is associated with normal variation in the blood oxygen saturation level, and when it is determined that the leading coefficient is smaller than the predetermined value, output a warning indicating that the blood oxygen saturation level of the user is getting worse.

Another object of the disclosure is to provide the monitoring device as described above for monitoring blood oxygen saturation level of a user.

Another object of the disclosure is to provide a machine-readable storage medium that is configured to implement the above-mentioned blood oxygen monitoring method.

According to one embodiment of the disclosure, the machine-readable storage medium includes instructions that, when executed b a monitoring device, cause a monitoring device to communicate with a blood oxygen saturation detector and to execute the above-mentioned method.

### BRIEF DESCRIPTION OF THE DRAWINGS

Other features and advantages of the disclosure will become apparent in the following detailed description of the embodiments with reference to the accompanying drawings, of which:

FIG. 1 is a flow chart illustrating steps of a method for monitoring blood oxygen saturation level of a user according to an embodiment of the disclosure;

FIG. 2 is a block diagram illustrating a monitoring system for monitoring blood oxygen saturation level of a user according to an embodiment of the disclosure;

FIG. 3 is a block diagram illustrating a monitoring system for monitoring blood oxygen saturation level of a user according to another embodiment of the disclosure; and

FIG. 4 exemplary illustrates a curve that fits values of blood oxygen saturation level according to an embodiment of the disclosure.

### DETAILED DESCRIPTION

Before the disclosure is described in greater detail, it should be noted that where considered appropriate, reference numerals or terminal portions of reference numerals have been repeated among the figures to indicate corresponding or analogous elements, which may optional have similar characteristics.

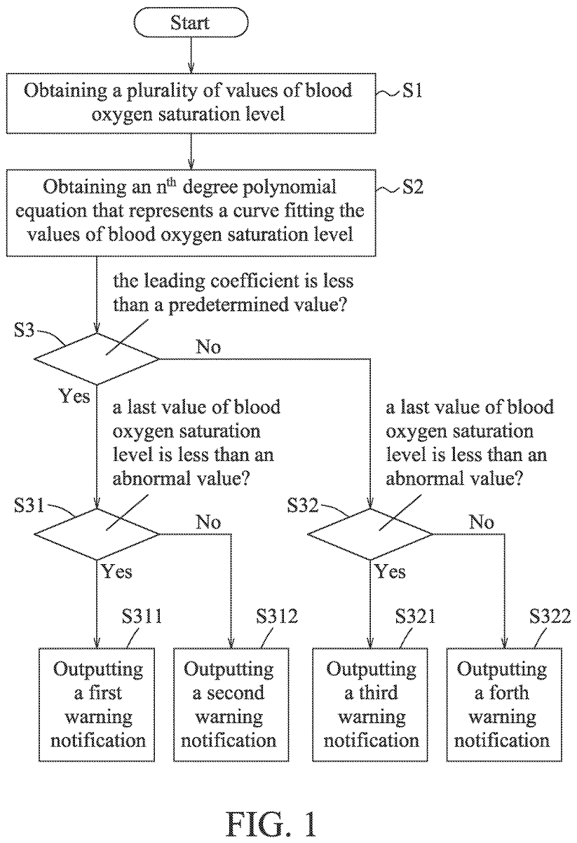

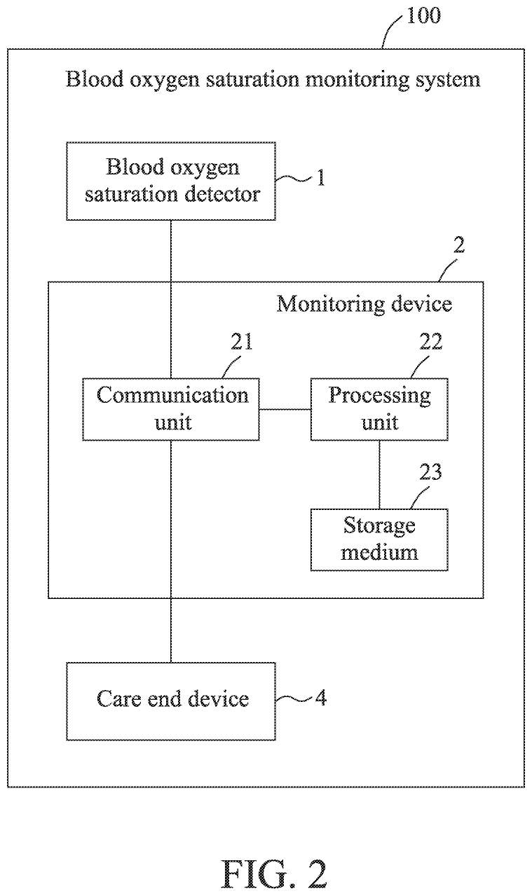

FIG. 1 is a flow chart illustrating steps of a method for monitoring blood oxygen saturation level of a user according to one embodiment of the disclosure. The method is implemented by a monitoring system 100 shown in FIG. 2.

Referring to FIG. 2, the monitoring system 100 includes a blood oxygen saturation detector 1, a monitoring device 2 communicating with the blood oxygen saturation detector 1, and a care end device 4 to be held by a caregiver.

The blood oxygen saturation detector 1 (e.g., a pulse oximeter) is worn by the user to periodically measure the blood oxygen saturation level of the user Generally, a normal value of blood oxygen saturation level ranges from 95% to 100%. More specifically, the blood oxygen saturation detector 1 may be controlled, by the monitoring device 2, to automatically measures the blood oxygen saturation level of the user and to send a value of the blood oxygen saturation level thus measured to the monitoring device 2 periodically, such as once every minute. Alternatively, the user may manually operate the blood oxygen saturation detector 1 in response to instructions from the monitoring device 2, so as to make the blood oxygen saturation detector 1 measure the blood oxygen saturation level of the user and send a value of the blood oxygen saturation level thus measured to the monitoring device 2 at predetermined moments of the day, for example, four times a day, respectively in the morning, at noon, in the evening, and before bedtime.

The monitoring device 2 may be, but not limited to, a mobile communication device (such as a smartphone, a tablet computer, etc.), and includes a communication unit 21, processing unit 22 electrically connected to the communication 21, and a storage medium 23 electrically connected to the processing unit 22. The monitoring device 2 communicates with the blood oxygen saturation detector via the communication unit through short-range wireless communication. For example, the communication unit 21 may include a short-range wireless communication module supporting a short-range wireless communication network using a wireless technology of Bluetooth® and/or Wi-Fi, etc., and/or the like. Similarly, the blood oxygen saturation detector 1 also includes a corresponding short-range wireless communication module (not shown) supporting a short-range wireless communication network using a wireless technology of Bluetooth® and/or Wi-Fi, etc., and/or the like. The storage medium 23 is a machine-readable storage medium, and stores instructions that, when executed by the processing unit 22, cause the processing unit 22 to query the blood oxygen saturation detector 1 for information related to blood oxygen saturation level and to execute steps described below.

The processing unit 22 may be embodied using a central processing unit (CPU), a microprocessor, a microcontroller, a single core processor, a multi-core processor, a dual-core mobile processor, a microprocessor, a microcontroller, a digital signal processor (DSP), a field-programmable gate array (FPGA), an application specific integrated circuit. (ASIC), a radio-frequency integrated circuit (RFIC), etc. The storage medium 23 may be embodied using, for example, random access memory (RAM), read only memory (ROM), programmable ROM (PROM), firmware, flash memory, etc. In some embodiments, the communication unit 21 may further include a mobile communication module supporting telecommunication using Long-Term Evolution (LTE), the third generation (3G) and/or fifth generation (5G) of wireless mobile telecommunications technology, and/or the like.

It noted that blood oxygen saturation detector 1 and the monitoring device 2 may be integrated as a single wearable electronic device to be worn by the user, such as a smartwatch.

The monitoring device 2 further communicates with the care end device 4 via short-range wireless communication or the Internet.

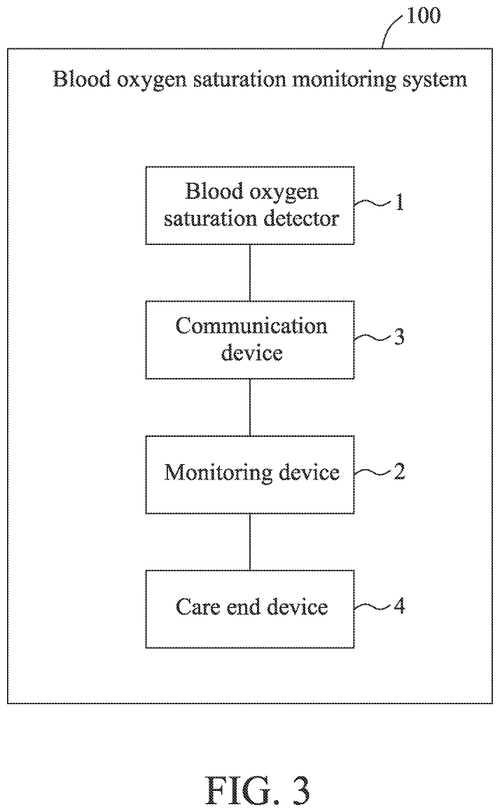

It is noted that, in some embodiments (see FIG. 3), the monitoring system 100 further includes a communication device 3 that communicates with the blood oxygen saturation detector 1 and the monitoring device 2. The blood oxygen saturation detector 1 communicates with the monitoring device 2 through the communication device 3. In one example, the blood oxygen saturation detector 1 has a built-in Bluetooth® module, the monitoring device 2 a remote sever, the communication device 3 a portable communication device (e.g., a smartphone, a tablet computer, etc.) having a built-in Bluetooth® module and is held by the user, and the blood oxygen saturation detector 1 communicates with the monitoring device 2 over the Internet through the portable communication device. In another example, the blood oxygen saturation detector 1 has a built-in Wi-Fi module, the monitoring device 2 is a remote server, the communication device is a wireless access point disposed in a space where the blood oxygen saturation detector 1 is located, and the blood oxygen saturation detector 1 communicates with the monitoring, device 2 over the Internet through the wireless access point.

The method for monitoring blood oxygen saturation level of a user according to one embodiment includes the following steps.

In step S1, the monitoring device 2 obtains a plurality of values of blood oxygen saturation level that were measured by the blood oxygen saturation detector 1. Specifically, the values of blood oxygen saturation level were measured in a time period of a predetermined duration. The time period may range from 4 hours to 48 hours in duration. In this embodiment, the predetermined duration is 48 hours. In this embodiment, the monitoring device 2 receive a value of blood oxygen saturation level from the blood oxygen saturation detector 1 periodically, such as once every minute, and store the received value therein. Once 48-hour worth of values have been stored therein, the monitoring device 2 retrieves these values in step S1.

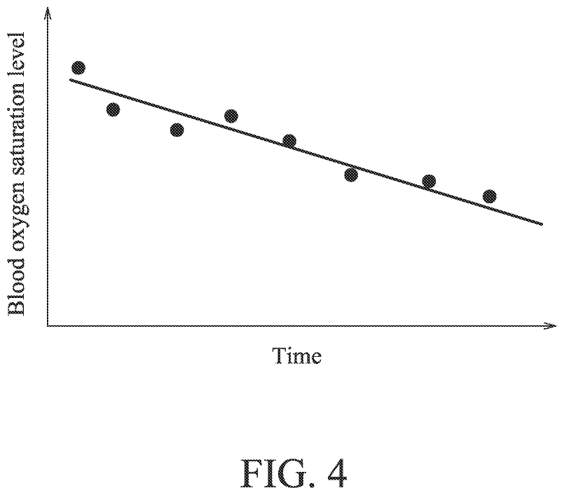

In step S2, the monitoring device 2 obtains an n^{th} degree polynomial equation in two variables that represents a curve which fits the values of blood oxygen saturation level obtained in step S1, where n is an integer equal to or greater than one. The n^{th} degree polynomial equation is obtained by curve fitting algorithm, also known as linear regression algorithm. As illustrated in FIG. 4, the curve shows the trend of change of the blood oxygen saturation level over time, and each point in the chart represents a measured value of blood oxygen saturation level. It is noted that the curve illustrated in FIG. 4 is a straight line, but the n^{th} degree polynomial equation may represent a curve that is a non-straight line, depending on the value or n.

For example, in the case where n is one, the monitoring device 2 obtains a first degree polynomial equation in two variables (i.e., Y=aX+b) to represent the curve that fits the values of blood oxygen saturation level measured in the 48-hour time period, where Y represents a value of blood oxygen saturation level, X represents time in seconds, a is a leading coefficient of the polynomial equation representing the slope of the curve, and b is a y-intercept of the polynomial equation. Furthermore, in the case where n is two, the monitoring device 2 obtains a second degree polynomial equation in two variables (i.e., Y=aX²+bX+c) to represent the curve that fits the values of blood oxygen saturation level measured in the 48-hour time period, where Y represents a value of blood oxygen saturation level, X represents time in seconds, a is a leading coefficient of the polynomial equation representing the slope of the curve, b is a second coefficient of the polynomial equation, and c is a y-intercept of the polynomial equation. The degree of the polynomial equation may be three or more depending on needs of practical application of the monitoring device 2, and may be determined by the user or a medical professional (e.g., the user's doctor). The degree of the polynomial equation may be determined by the values of blood oxygen saturation level being observed, and preferably is one or two.

In step S3, the monitoring device 2 determines whether the leading coefficient of the n^{th} degree polynomial equation is smaller than a predetermined value that is associated with a normal variation in blood oxygen saturation level. When it is determined that the leading coefficient is smaller than the predetermined value, the flow goes to step S31; otherwise, the flow goes to step S32. The leading coefficient of the n^{th} degree polynomial equation being smaller than the predetermined value is representative of the blood oxygen saturation level of the user getting worse. The predetermined value is a slope of the normal variation in blood oxygen saturation level in time period of the predetermined duration.

For example, what is generally considered a normal variation in blood oxygen saturation level within a 48-hour period is a variation that at most exhibits a decline from 100% to 96%, and thus the predetermined value 1 calculated by (961-100%)/(48*60*60 seconds); that is, the predetermined value is a negative value. Therefore, the leading coefficient the n^{th} degree polynomial equation being smaller than the predetermined value indicates that the variation in blood oxygen saturation level of the user is abnormal and that the blood oxygen saturation level of the user is getting worse.

When it is determined that the leading coefficient is smaller than the predetermined value, in step S31, the monitoring device 2 further determines whether a last one of the values of blood oxygen saturation level smaller than an abnormal value that represents severe low level or blood oxygen saturation. For example, the abnormal value is 90%. The flow goes to step S311 when it is determined that the last one of the values of blood oxygen saturation level is smaller than the abnormal value, and goes to step S312 when otherwise.

When it is determined that the leading coefficient is smaller than the predetermined value (i.e., the blood oxygen saturation of the user is getting worse) and the last one of the values of blood oxygen saturation level is smaller than the abnormal value, in step S311, the monitoring device 2 outputs a first warning notification (e.g., making a warning, sound and showing a warning message) to warn (or wake) the user and to instruct the user to take appropriate action (e.g., making a phone call or sending a message to a medical professional at the quarantine facility where the user is located). At the same time, the monitoring device 2 also transmits a signal containing the first warning notification and the values of blood oxygen saturation level to the care end device 4 for record keeping, and the care end device 4 outputs the first warning notification to instruct the caregiver to take appropriate action (e.g., sending staff to check on the user or asking the user about his/her condition via a video call).

When it is determined that the leading coefficient is smaller than the predetermined value and the last one of the values of blood oxygen saturation level is not smaller than (i.e., greater than or equal to) the abnormal value, in step S312, the monitoring device 2 outputs a second warning notification to instruct the user to pay attention to subsequent variation in the blood oxygen saturation level. At the same time, the monitoring device 2 also transmits a signal containing the second warning notification and the values of blood oxygen saturation level to the care end device 4 for record keeping, and the care end device 4 outputs the second warning notification to notify the caregiver to keep an eye on the development of user's blood oxygen saturation level.

When it is determined that the leading coefficient is not smaller than the predetermined value, in step S32, the monitoring device 2 further determines whether the last one of the values of blood oxygen saturation level is smaller than the abnormal value. The flow goes to step S321 when it is determined that the last one of the values of blood oxygen saturation level is smaller than the abnormal value, and goes to step S322 when otherwise.

When it is determined that the leading coefficient is not smaller than the predetermined value and the last one of the values of blood oxygen saturation level is smaller than the abnormal value, step S321, the monitoring device 2 outputs a third warning notification to instruct the user to pay attention to subsequent variation in the blood oxygen saturation level. At the same time, the monitoring device 2 also transmits a signal containing the third warning notification and the values of blood oxygen saturation level to the care end device 4 for record keeping, and the care end device 4 outputs the third warning notification to notify the caregiver to keep an eye on the development of the blood oxygen saturation level of the user.

When it is determined that the leading coefficient is not smaller than the predetermined value and the last one of the values of blood oxygen saturation level is not smaller than the abnormal value, in step S322, the monitoring device 2 outputs a normal notification to notify the user that the blood oxygen saturation level of the user is normal and does not exhibit a worsening trend. At the same time, the monitoring device 2 also transmits a signal containing the normal notification to the care end device 4 for record keeping, and the care end device 4 outputs the normal notification to notify the caregiver that the blood oxygen saturation level of the user is normal and does not exhibit a worsening trend.

It is noted that, in some embodiments, the care end device 4 may be omitted.

It is noted that, in some embodiments where the monitoring device 2 is a portable communication device or is integrated into a wearable electronic device worn by the user, the above-mentioned notifications will be directly output by the monitoring device 2. Alternatively, in some embodiments where the monitoring device 2 is a remote server, contents of the above-mentioned notifications be output to a portable communication device (e.g., the communication device 3) or the blood oxygen saturation detector 1.

The method of the disclosure may be repeated to continuously monitor the blood oxygen saturation level of the user.

To sum up, b the ems of the disclosure provide a method, a monitoring system 100 and a monitoring device 2 for monitoring blood oxygen saturation level of a user. The monitoring device 2 obtains a plurality of values of blood oxygen saturation level from the blood oxygen saturation detector 1, and obtains the n^{th} degree polynomial equation, and determines whether the leading coefficient of the n^{th} degree polynomial equation is smaller than the predetermined value so as to determine whether the blood oxygen saturation level is getting worse. By virtue of the method and the monitoring device 2, variation in the blood oxygen saturation level of the user can be monitored anytime, and a warning notification can be issued immediately without waiting for a doctor or a relevant medical professional to make a judgment on site.

In the description above, for the purposes of explanation, numerous specific details have been set forth in order to provide a thorough understanding of the embodiments. It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometime grouped together in a single embodiment, figure, or description thereof for the purpose of streamlining the disclosure and aiding in the understanding, or various inventive aspects, and that one or more features or specific details from one embodiment may be practiced together with one or more features or specific details from another embodiment, where appropriate, in the practice of the disclosure.

While the disclosure has been described connection with what are considered the exemplary embodiments, it is understood that this disclosure is not limited to the disclosed embodiments but is intended to cover various arrangements included within the spirit and scope of the broadest interpretation so as to encompass all such modifications and equivalent arrangements.

What is claimed is:

1. A method for monitoring blood oxygen saturation level of a user, the method to be implemented by a monitoring device communicating with a blood oxygen saturation detector, the blood oxygen saturation detector being worn by the user to periodically measure the blood oxygen saturation level of the user, the method comprising steps of:
obtaining a plurality of values of blood oxygen saturation level from the blood oxygen saturation detector, the values of blood oxygen saturation level being detected during in a time period of a predetermined duration;
obtaining an n^{th} degree polynomial equation in two variables that represents a curve which fits the values of blood oxygen saturation level, where n is an integer equal to or greater than one;
determining whether a leading coefficient of the n^{th} degree polynomial equation is smaller than a predetermined value that is associated with a normal variation in blood oxygen saturation level; and
when it is determined that the leading coefficient is smaller than the predetermined value, outputting a warning indicating that the blood oxygen saturation level of the user is getting worse.

2. The method of claim 1, wherein the leading coefficient represents a slope of the curve that fits the values of blood oxygen saturation level, and the predetermined value represents a slope of the normal variation in blood oxygen saturation level during a time period of the predetermined duration.

3. The method of claim 1, wherein the step of outputting a warning notification includes:
determining whether a last one of the values of blood oxygen saturation level is smaller than an abnormal value that represents severe low level of blood oxygen saturation; and
outputting a first warning notification to serve as the warning when it is determined that the last one of the values of blood oxygen saturation level is smaller than the abnormal value.

4. The method of claim 3, wherein the step of outputting a warning further includes outputting a second warning notification to serve as the warning when it determined that the last one of the values of blood oxygen saturation level is not smaller than the abnormal value.

5. The method of claim 1, further comprising steps of:
when it is determined that the leading coefficient is not smaller than the predetermined value, determining whether a last one of the values of blood oxygen saturation level is smaller than an abnormal value that represents severe low level of blood oxygen saturation; and
outputting a first notification when it is determined that the last one of the values of blood oxygen saturation level is smaller than the abnormal value.

6. The method of claim 5, further comprising a step of outputting a second notification when it is determined that the last one of the values of blood oxygen saturation level is not smaller than the abnormal value.

7. The method of claim 1, wherein the predetermined duration ranges from 4 hours to 48 hours.

8. A monitoring system for monitoring blood oxygen saturation level of a user, comprising:
a blood oxygen saturation detector adapted to be worn by the user, and configured to periodically measure the blood oxygen saturation level of the user and to output a value of blood oxygen saturation level for each measurement of the blood oxygen saturation level; and
a monitoring device configured to
communicate with said blood oxygen saturation detector to obtain a plurality of values of blood oxygen saturation level from said blood oxygen saturation detector, the values of blood oxygen saturation level being measured in a time period of a predetermined duration,
obtain an n^{th} degree polynomial equation in two variables that represents a curve which fits the values of blood oxygen saturation level, where n is an integer equal to or greater than one,
determine whether a leading coefficient of the n^{th} degree polynomial equation is smaller than a predetermined value that is associated with a normal variation in blood oxygen saturation level, and
when it is determined that the leading coefficient is smaller than the predetermined value, output a warning indicating that the blood oxygen saturation level of the user is getting worse.

9. The monitoring system of claim 8, wherein the leading coefficient represents a slope of the curve that fits the values of blood oxygen saturation level, and the predetermined value represents a slope of the normal variation in blood oxygen saturation level in a time period of the predetermined duration.

10. The monitoring system of claim 8, wherein said monitoring device is configured to output the warning
determining whether a last one of the values of blood oxygen saturation level is smaller than an abnormal value that represents severe low level of blood oxygen saturation; and
outputting a first warning notification to serve as the warning when it is determined that the last one of the values of blood oxygen saturation level is smaller than the abnormal value.

11. The monitoring system of claim 10, wherein said monitoring device is configured to output the warning by outputting a second warning notification to serve as the warning when it is determined that the last one of the values of blood oxygen saturation level is not smaller than the abnormal value.

12. The monitoring system of claim 8, wherein said monitoring device is further configured to:
when it is determined that the leading coefficient is not smaller than the predetermined value, determine whether a last one of the values of blood oxygen saturation level is smaller than an abnormal value that represents severe low level of blood oxygen saturation; and
output a first notification when it is determined that the last one of the values of blood oxygen saturation level is smaller than the abnormal value.

13. The monitoring system of claim 12, wherein said monitoring device is further configured to output a second notification when it is determined that the last one of the values of blood oxygen saturation level is not smaller than the abnormal value.

14. The monitoring system of claim 8, wherein the predetermined duration ranges from 4 hours to 48 hours.

15. The monitoring system of claim 8, further comprising a communication device which communicates with said blood oxygen saturation detector and said monitoring device, wherein said monitoring device is a remote server, and said blood oxygen saturation detector communicates with said monitoring device over the Internet through said communication device.

16. The monitoring system of claim 8, wherein said blood oxygen saturation detector and said monitoring device are integrated into a wearable electronic device to be worn by the user.

17. A monitoring device for monitoring blood oxygen saturation level of a user, comprising:
a communication unit configured to communicate with a blood oxygen saturation detector that is worn by the user to periodically measure the blood oxygen saturation level of the user;
a processing unit electrically connected to said communication unit; and
a storage medium electrically connected to said processing unit, and storing instructions that when executed by said processing unit cause said processing unit to
obtain a plurality of values of blood oxygen saturation level from the blood oxygen saturation detector via said communication unit, the values or blood oxygen saturation level being measured in a time period of a predetermined duration,
obtain an n^{th} degree polynomial equator in two variables that represents a curve which fits the values of blood oxygen saturation level, where n is an integer equal to or greater than one,
determine whether a leading coefficient of the n^{th} degree polynomial equation is smaller than a predetermined value that associated with a normal variation in blood oxygen saturation level, and
when it is determined that the leading coefficient is smaller than the predetermined value, output a warning indicating that the blood oxygen saturation level of the user is getting worse.

18. The monitoring device of claim 17, wherein the leading coefficient represents a slope of the curve that fits the values of blood oxygen saturation level, and the predetermined value represents a slope of the normal variation in blood oxygen saturation level in the time period.

19. The monitoring device of claim 17, wherein said processing unit is configured to output the warning by:
determining whether a last one of the values of blood oxygen saturation level is smaller than an abnormal value that represents severe low level of blood oxygen saturation; and
outputting a first warning notification to serve as the warning when it is determined that the last one of the values of blood oxygen saturation level is smaller than the abnormal value.

20. The monitoring device of claim 19, wherein said processing unit is configured to output the warning by outputting a second warning notification to serve as the warning when it is determined that the last one of the values of blood oxygen saturation level is not smaller than the abnormal value.

21. The monitoring device of claim 17, wherein said processing unit is further configured to:
when it is determined that the leading coefficient is not smaller than the predetermined value, determine whether a last one of the values of blood oxygen saturation level is smaller than an abnormal value that represents severe low level of blood oxygen saturation; and
output a first notification when it is determined that the last one of the values of blood oxygen saturation level is smaller than the abnormal value.

22. The monitoring device of claim 21, wherein said processing unit is further configured to output a second notification when it is determined that the last one of the values of blood oxygen saturation level is not smaller than the abnormal value.

23. The monitoring device of claim 17, wherein the predetermined duration ranges from 4 hours to 48 hours.

24. A machine-readable storage medium, comprising instructions that, when executed by a monitoring device, cause the monitoring device to communicate with a blood oxygen saturation detector and to execute the method of claim 1.
